(19) United States
(12) Patent Application Publication
(10) Pub. No.: US 2006/0272651 A1
(43) Pub. Date: Dec. 7, 2006

(54) DIVERSION BOARD/DIVERSION SHIELD

(75) Inventor: STEPHANIE ORTEL, Collingswood, NJ (US)

Correspondence Address: RATNERPRESTIA, P O BOX 980, VALLEY FORGE, PA, 19482-0980, US

(73) Assignee: THE CHILDREN'S HOSPITAL OF PHILADELPHIA, Philadelphia, PA (US)

(21) Appl. No.: 11/421,931

(22) Filed: Jun. 2, 2006

Related U.S. Application Data
(60) Provisional application No. 60/687,231, filed on Jun. 3, 2005.

(51) Int. Cl. A61B 19/00

(52) U.S. Cl. 128/869

(57) ABSTRACT

A diversion board includes a flexible supportive plastic board, or the like, measuring about 2 to 3 feet in height and 3 to 4 feet in width. The board includes an aperture through its interior or at its bottom for an arm or body limb to fit through. One side of the board is designed to face the patient, for example a child patient, and includes pleasant decals, cartoon characters or a diversionary toy (e.g., press music device, electronic game, mechanical game, sensory display, etc.). The board is designed to stand on a horizontal plane, such as a treatment table or floor, and is also light enough that a parent or guardian can hold the board up as they hold or cuddle a child. The diversion board blocks the view between the child and the injection site, and allows the child to look at something more pleasant, thereby reducing the natural stress and apprehension of the child. The board is useful in treatment rooms, labs and home care, and is easy to store and clean.

### BACKGROUND OF THE INVENTION

1. Field of Invention

This invention relates to devices that help to relieve a patient's anxiety during a medical treatment. In particular, this invention relates to a decorative device that diverts a patient's attention from a treatment area to the device during the medical treatment.

2. Description of Related Art

Visits to a hospital or doctor's office can be a scary and anxiety provoking experience for many patients, particularly children. The anxiety of entering a new environment full of new faces, foreign instruments and ominous medical equipment can stimulate a sympathetic nervous response in patients leading to objective physical stress, for example, increased heart rate, increased blood pressure, sweating, emotional instability, and changes in arterial blood gases. Many doctors find that it is advantageous to relieve these fears and anxieties before and throughout a medical treatment or procedure. This allows the patient to feel more at ease during the exam, which in turn allows for a safer and more reliable treatment.

Child patients have heightened anxieties and less emotional control than adult patients. Because children have a wider range of psychological responses to their medical treatment, instilling a good mental attitude in the patient is critical when treating children. A child is usually very anxious about the friendliness and care he or she will receive during a medical treatment, and this apprehension may hinder both treatment and recovery. During a medical treatment, (e.g., venipuncture, IV introduction), children or for that matter many adults, have a natural tendency to look at a task being performed, as they are curious and want to see the source of discomfort at the treated area. Currently, people are used to divert the patient. However, patients, and in particular, children, become even more agitated if they are restrained by people and as they witness an injection needle, syringe, and site of blood.

The importance of a child's psychological security during medical treatment is well recognized. U.S. Pat. No. 6,520,639 discloses a decorative cover for medical equipment for reducing some of the intimidation created by medical equipment. The cover is configured to receive a medical device in a shape to portray an animal, plant, vehicle or building. U.S. Pat. No. 4,976,646 discloses a medicine pal toy which engages a finger or a liquid medicine dispenser acting as a lowered jaw for the toy. These patents, however, primarily address attachments for medical instruments that are attractive to a child, making the instruments less threatening.

U.S. Pat. No. 6,237,688 discloses a voting convertible screen/display made from rigid, structural material comprising a floor and three walls positionable and free standing on a support surface. U.S. Pat. No. 6,641,522 discloses a fabric frame display member mounted on a stand that shows serene landscape scenes for relaxing a person in a stressful environment, such as a health care or hospital setting. However, neither of these patents address an approach for blocking the view between the patient and the treatment site or area and diverting the patient's attention to something more pleasant.

It would thus be beneficial to provide an approach to relieve a patient's anxiety during a medical treatment by blocking the patient's view of the treatment area and diverting the patient's attention. All references cited herein are incorporated herein by reference in their entireties.

### BRIEF SUMMARY OF THE INVENTION

The preferred embodiments of the invention include a diversion board having an aperture through its interior or an opening at its bottom for a body part (e.g., arm, leg, torso, limb) to fit through. A diversion board is preferably a flexible plastic board measuring about 2 to 3 feet in height and 3 to 4 feet in width. One side of the board is designed to face the patient and includes pleasant decals, cartoon characters and/or a diversionary toy (e.g., press music device, sensory display, picture, book, electronic game, mechanical game, telephone, mirror, light display). The board is designed to stand on a horizontal plane, such as a treatment table or floor, and is also light weight so that an adult can hold the board up with one hand as they hold or cuddle the patient.

In a preferred embodiment, the diversionary board includes a folding convertible self-standing shield and a pass-through integrated into the shield. The shield is formed of a light weight bendable structural material and has a first side, a second side, a top edge, a bottom edge, a first side edge and a second side edge. The first side includes a diversionary toy located thereon and adapted to face a patient. The pass-through is adjacent the bottom edge and adapted to receive a limb of the patient. The shield is preferably non-transparent and blocks the patient's view of the treatment site of the limb while the diversionary tool attracts the patient's attention during a medical treatment at the treatment site as the patient's limb is extended through the pass-through. The pass-through is preferably located within the shield or along the bottom edge of the shield.

The preferred embodiments of the invention also include a method of using a diversion board to divert the attention of a patient away from a limb of the patient being medically treated. The method includes placing the diversion board over or about the limb between the eyes of the patient and a distal area of the limb that will be accessed for the medical treatment, diverting the attention of the patient with a diversionary tool located on the diversion board and facing the patient, and removing the diversionary board from the patient after the medical treatment is completed.

Further scope of applicability of the present invention will become apparent from the detailed description given hereinafter. However, it should be understood that the detailed description and specific examples, while indicating preferred embodiments of the invention, are given by way of illustration only, and that the invention is not limited to the precise arrangements and instrumentalities shown, since the invention will become apparent to those skilled in the art from this detailed description.

### BRIEF DESCRIPTION OF SEVERAL VIEWS OF THE DRAWINGS

The following detailed description of preferred embodiments of the invention will be better understood when read in conjunction with the following drawings, in which like reference numerals designate like elements and wherein:

FIG. 1 is a plan view of a diversion board in accordance with a preferred embodiment of the invention;

FIG. 2 is a plan view of a second diversion board in accordance with another preferred embodiment of the invention; and

FIG. 3 is an opposite plan view showing an application of the diversion board in accordance with the preferred embodiments of the invention.

### DETAILED DESCRIPTION OF THE INVENTION

While not being limited to a particular theory, a diversion board according to the preferred embodiments is a folding convertible self-standing board or shield formed of a light weight bendable non-transparent structural material. The board includes a diversionary toy that in operation faces, and attracts the attention of a patient, and in particular, a child. The inventor recognized that blocking a child's view of a medically treated area (e.g., elbow, wrist, hand, foot), and diverting the child's attention helps to alleviate the child's apprehension and allows the medical staff to treat the patient more effectively and without having to deal with an unruly patient.

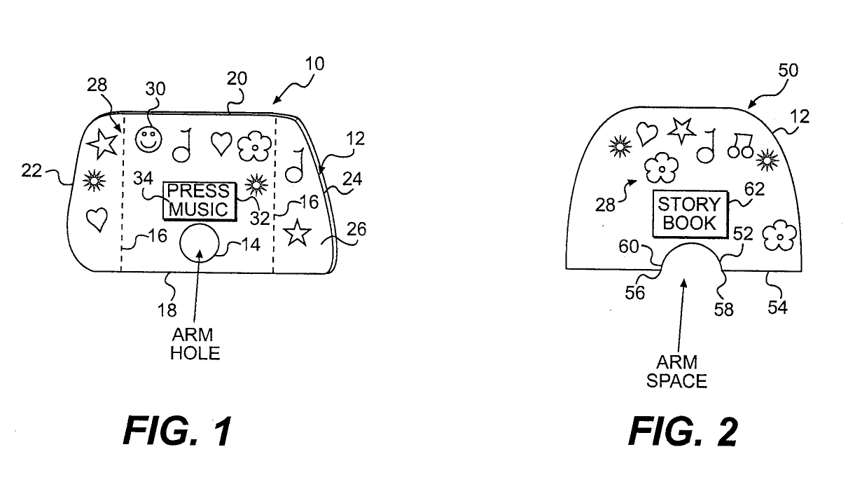

Diversion boards in accordance with the preferred embodiments are shown in the figures. For example, FIG. 1 shows a diversion board 10 having a shield 12 and a pass-through 14. The shield 12 is formed from a light weight bendable material (e.g., plastic board), preferably about 2 to 3 feet in height and 3 to 4 feet in width. The shield 12 is bendable, preferably along bend lines 16 which permit the shield to fold for easy transportation. In other words, the shield 12 is preferably a single piece of rigid structural material having flexible portions, for example, the bend lines 16 that permit the shield to easily fold or bend to form a self-standing configuration along its bottom edge 18. The bend lines 16 are not critical to the invention, as they are one example of an approach to help the diversion board stand on its own. Alternative approaches are available that enable the diversion board 10 to stand, such as having the side walls of the board flexible to roll or curl inward so as to create a planar bottom edge section. Another alternative is to attach legs to the board 10 that project outward, preferably in a direction normal to the shield 12 and hold the board 10 upright when placed on a substantially horizontal surface (e.g., floor, table top). One obvious advantage of these designs lies in the ease by which the board 10 can be used and transported, since the board 10 does not consist of numerous pieces which must be disassembled and reassembled with each break down and set up.

Still referring to FIG. 1, the pass-through 14 is an aperture located within the shield 12. While not being limited to a particular theory, the pass-through 14 is preferably round (e.g., circular, oval) and is sized to accommodate an arm of a patient. That is, the pass-through 14 is sized to allow a patient to enter his or her arm through the pass-through up to the patient's shoulder. In other preferred embodiments, the pass-through 14 is configured to also allow a patient's leg to extend through. During operation of the diversion board 10, when the patient's limb is extended through the pass-through 14, the board preferably blocks the patient's view of an area (e.g., elbow, wrist, hand) of the extended limb distal to the board that is accessed for medical treatment (i.e., venipuncture, IV). In addition to the bottom edge 18, the shield is defined at its circumference by a top edge 20, a first side edge 22 and a second side edge 24. The edges combine to define a substantially rectangular shaped shield 12 with rounded edges between each of the top edge 20, first side edge 22, bottom edge 18, and second side edge 24. It is understood that the shape of the shield is not critical to the invention, as shields having shapes that are substantially triangular, trapezoidal, or any other polygonal shape are within the scope of the invention.

As can be seen in FIG. 1, the shield 12 has a first side 26 that includes a diversionary toy 28 located thereon and adapted to face the patient. The diversionary toy 28 is a sensory display that attracts a patient's attention. Preferably, the sensory display includes at least one picture 30 that is attractive to the patient, and in particular, to a child. The picture 30 can include shapes or alphanumeric symbols, scenery, a mirror, a book or any other artistic design attractive to the patient. As can best be seen in FIG. 1, the diversionary toy 28 also includes an interactive device, for example, a music device 32. The music device 32 has a cover that includes an activation button 34. Moreover, the music device 32 preferably includes speakers and electronics necessary to create and amplify a musical sound, as is readily understood by a skilled artesan. In operation, the patient presses the button 34 which commences the playing of a song by the music device 32. When the song ends, the button 34 of the music device 32 can be depressed again to play another song. As an alternative to a music device, the diversionary toy may also include interactive games or devices (e.g., electronic game, mechanical game, telephone) that can be operate by the patient.

FIG. 2 shows an alternative diversion board 50 to the diversion board 10 shown in FIG. 1. While not being limited to a particular theory, the diversion board 50 is substantially similar to the diversion board 10 but has some noteworthy differences. In particular, the diversion board 50 includes a pass-through 52 that is an aperture along the bottom edge 54 of the board 50. The pass-through 52 is shown as a cut out of the bottom edge 54 to allow the board 50 to be placed over the patient's arm, leg or desired body part and block the patient's view of the area distal to the board that is being treated. The most significant difference between the diversion board 10 shown in FIG. 1 and the diversion board 50 shown in FIG. 2 is the placement of the pass-through. The pass-through 14 of the diversion board 10 is completely enclosed within and surrounded by the shield 12. This structural design requires that the patient insert his or her arm or leg through the pass-through 14 for treatment.

As a preferred alternative approach, the pass-through 52 shown in FIG. 2 is generally a semi-circular cut-out from shoulder 56 to shoulder 58 of the bottom edge 54. That is, the pass-through 52 extends into the shield 12 to form a generally semi-circular or U-shaped rim 60 that can be fitted on the patient in a variety of ways. For example, the patient can insert his or her limb through the pass-through 52 in a manner similar to the manner discussed above for the diversion board 10. In addition, the board 50 can be placed over the patient's limb with the limb extended through the pass-through 52. Preferably, under either approach, the bottom edge 54 can be placed on and stand up from a substantially horizontal planar surface (e.g., table top, floor). However, since the diversion boards 50 and 10 are light weight, they can also be held by a parent or medical staff person and still operate for their intended purpose of blocking the view of the patient from the treatment site and divert the patient's attention to the diversionary toy on the shield.

While not being limited to a particular theory, the shield 12 includes a storybook 62 as its diversionary toy. It is understood, of course, that other diversionary toys may be placed in lieu of or in addition to the storybook 62 to divert the patient's attention. The storybook 62 can be any of many types of books, including interactive electronic storybooks, traditional story books and coloring books. Other diversionary toys may be used as well, including a mechanical game, telephone, mirror, picture, music device, or light display as discussed above for the diversion board 10.

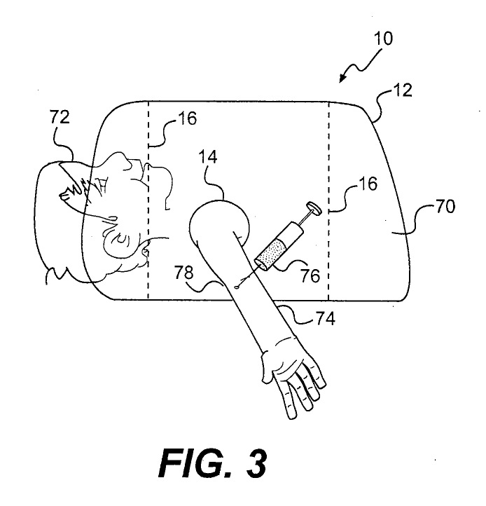

FIG. 3 is an illustration of the diversion board 10 during operation from the viewpoint of a person administering the treatment. The diversion board 10 includes a second side 70, which is opposite the first side 26 of the board. As can be seen in FIG. 3, a patient 72 has his or her arm 74 extended through the pass-through 14 of the diversion board 10 for a medical treatment (e.g., injection, getting a blood sample) with a syringe 76 at the elbow area 78 of the arm. During normal use of the diversion board 10, the second side 70 faces away from the patient's face and toward the treatment area (e.g., elbow area 78) distal to the board.

Still referring to FIG. 3, the second side 70 of the exemplary diversion board 10 does not include a diversionary toy as discussed above. That is, a diversionary toy is not required on the second side 70 since the second side normally does not face the patient and is generally not used to attract the patient's attention. However, it is understood that, if desired, the second side 70 could include such diversionary toys as discussed above. In this manner, the diversionary board 10 becomes reversible in that either the first side 26 or the second side 70 could face the patient during operation and divert the patient's attention away from the treatment site. Under this approach, the diversionary board 10 could include different diversionary toys on its first and second sides, thereby giving the patient a choice of different diversionary toys for interaction. As discussed above, the diversionary board 10, and for that matter, the diversionary board 50, preferably includes bend lines 16 to ease the bending or folding properties of the board, and to help the board 10 stand on its own or fold compactly for transport or storage.

While not being limited to a particular theory, the diversion boards of the preferred embodiments are made of a material that enables the board to stand securely on its bottom edge and bend as needed to help the board stand or fold. The preferred material is a plastic that is flexible yet rigid to make the board self standing. While plastic is the preferred material, it is understood that other materials may be used in the diversion board, including cardboard, that are light weight and structural supportive. The first side 26 and second side 70 may also include a plastic or clear coat cover to help protect the board from moisture, puncture or markings from a writing instrument (e.g., pencil, pen, crayon, marker). The protective covering or layer is preferably liquid resistant and can be wiped clean during or after use.

The diversion boards of the preferred embodiments can be used during numerous medical procedures and at numerous settings as readily understood by a skilled artisan. Examples of the numerous procedures include but are not limited to venipuncture—including IV placement, blood draws, injections (e.g., intramuscular, intravenous, subcutaneous) for sedation, vaccination, chemotherapy, botox treatment (e.g., for spasticity), etc.; biopsy; wound care; burn treatment; electromyography (EMG); or other medical treatment. While not being limited to a particular theory, exemplary settings for using the preferred diversion board include hospitals (e.g., treatment rooms, sedation rooms, emergency rooms), clinics, doctor's offices, laboratories, home, mobile units, or other locations where patient care is given. Moreover, the preferred diversion boards can be used in conjunction with other pain or stress reducing strategies, including but not limited to topical anesthetics, mild sedatives, acupuncture, massage, breathing exercises, audio, lighting and video.

It will be appreciated by those skilled in the art that changes could be made to the embodiments described above without departing from the broad inventive concept. For example, the diversionary toy may be placed on a layer of the shield that is not structurally supportive to stand on its own, for example, a thin sheet of paper or photograph. However, in this embodiment, the diversionary toy would be one of several layers of the board, with other layers being structurally supportive to keep the board in a self-standing configuration as desired. In particular, the protective layer or layers may be formed from a plastic that allows the patient to see through the cover to the diversionary toy and sufficiently supportive to keep the board in a self-standing orientation. It is understood, therefore, that this invention is not limited to the particular embodiments disclosed, but it is intended to cover modifications within the spirit and scope of the present invention. Without further elaboration, the foregoing will so fully illustrate my invention that others may, by applying current or future knowledge, readily adapt the same for use under various conditions of service.

What is claimed is:

1. A diversion board, comprising:
a folding convertible self-standing shield having a light-weight bendable structural material, said shield having a first side, a second side, a top edge, a bottom edge, a first side edge and a second side edge, said first side having a diversionary toy located directly thereon and adapted to face a patient; and
a pass-through integrated into said folding convertible shield adjacent said bottom edge, said pass-through adapted to receive a limb of the patient, said shield being adapted to block the patient's view of the limb and said diversionary toy being adapted to attract the patient's attention while the patient's limb is extended through said pass-through.

2. The diversion board of claim 1, wherein said pass-through is an aperture within said shield.

3. The diversion board of claim 1, wherein said pass-through is an aperture along said bottom edge of said shield.

4. The diversion board of claim 1, wherein said diversionary toy includes a sensory display.

5. The diversion board of claim 4, wherein said sensory display includes a picture.

6. The diversion board of claim 4, wherein said sensory display includes a music device.

7. The diversion board of claim 4, wherein said sensory display includes alphanumeric symbols.

8. The diversion board of claim 4, wherein said sensory display includes a book.

9. The diversion board of claim 4, wherein said sensory display includes an electronic game.

10. The diversion board of claim 4, wherein said sensory display includes a mechanical game.

11. The diversion board of claim 4, wherein said sensory display includes a telephone.

12. The diversion board of claim 4, wherein said sensory display includes a mirror.

13. The diversion board of claim 4, wherein said sensory display includes a light.

14. The diversion board of claim 1, wherein said folding convertible self-standing shield is nontransparent.

15. A method of using a diversion board to divert the attention of a patient away from a limb of the patient being medically treated, the diversion board having a folding convertible self-standing shield formed from a light-weight bendable structural material, a first side of the shield having a diversionary toy, the diversionary board also having a pass-through integrated into the folding convertible shield adjacent a bottom edge of the shield, the method comprising:
placing the diversion board about a first area of the limb between the eyes of the patient and a second area of the limb distal the first area and predetermined for a medical treatment;
diverting the attention of the patient with a diversionary toy located on the folding convertible self-standing shield and facing the patient, and
after the medical treatment at the second area, removing the diversion board from the patient.

16. A method of using a diversion board to divert the attention of a patient away from a limb of the patient being medically treated, the diversion board having a folding convertible self-standing shield formed from a light-weight bendable structural material, a first side of the shield having a diversionary toy, the diversionary board also having a pass-through integrated into the folding convertible shield adjacent a bottom edge of the shield, the method comprising:
placing the diversion board over a first area of the limb between the eyes of the patient and a second area of the limb distal the first area and predetermined for a medical treatment;
diverting the attention of the patient with a diversionary toy located on the folding convertible self-standing shield and facing the patient, and
after the medical treatment at the second area, removing the diversion board from the patient.
